(19) United States
(12) Patent Application Publication
(10) Pub. No.: US 2025/0388880 A1
(43) Pub. Date: Dec. 25, 2025

(54) USE OF RAP2C IN BREEDING OF BLCATTLE BLACK CATTLE

(71) Applicant: Qingdao Agricultural University, Qingdao (CN)

(72) Inventors: Yajuan DONG, Qingdao (CN); Xiuping MIAO, Qingdao (CN); Ruili LIU, Qingdao (CN)

(21) Appl. No.: 19/087,262

(22) Filed: Mar. 21, 2025

(30) Foreign Application Priority Data
Jun. 25, 2024 (CN) 202410825324.2

(51) Int. Cl. C12N 9/14; C12N 15/85; C12Q 1/6888

(52) U.S. Cl. CPC C12N 9/14; C12N 15/85; C12Q 1/6888; C12Y 306/05002; C12Q 2600/158

(57) ABSTRACT

Provided is the use of RAP2C in the breeding of Blcattle black cattle, belonging to the technical field of molecular biological breeding. RAP2C can be used for the breeding of Blcattle black cattle, and individuals having a high RAP2C content are screened as high-quality calves and alternative breeding cattle. In the present disclosure, it is proved by means of experiments that RAP2C can promote the differentiation of vascular endothelial progenitor cells to regulate placental angiogenesis, enhance placental blood flow and increase placental nutrient transport efficiency in Blcattle black cattle, thereby regulating calf birth weight. The present disclosure provides a new direction for the breeding of high-quality beef cattle of Blcattle black cattle.

### CROSS REFERENCE TO RELATED APPLICATION

The present application claims the right of priority for Chinese patent application No. CN2024108253242, filed with the China National Intellectual Property Administration on Jun. 25, 2024 and entitled “USE OF RAP2C IN BREEDING OF BLCATTLE BLACK CATTLE”, which is incorporated herein by reference in its entirety.

### REFERENCE TO SEQUENCE LISTING

A computer readable XML file entitled “GWP20250100144”, that was created on Mar. 6, 2025, with a file size of about 17, 758 bytes, contains the sequence listing for this application, has been filed with this application, and is hereby incorporated by reference in its entirety.

### TECHNICAL FIELD

The present disclosure belongs to the technical field of molecular biological breeding, and specifically relates to the use of RAP2C in the breeding of Blcattle black cattle.

### BACKGROUND

The information disclosed in the part of background only aims to enhance the understanding of general background of the present disclosure, should not be considered to admit or suggest in any form that the information constitutes the related art that has been known to those of ordinary skill in the art.

The placenta is a temporary organ unique to the mother during pregnancy. A mother provides nutrients such as oxygen, glucose, amino acids and fatty acids to a fetus through the placenta, and the fetus also excretes metabolic waste through the placenta. It can be seen that the growth and development of a fetus depend on the function of the placenta and the ability of the placenta to transport nutrients to the fetus. Placental blood vessels are the guarantee to ensure a fetus to obtain nutrients and oxygen, and placental angiogenesis disorders will seriously affect the growth and development of the fetus. Production practices demonstrate that calf birth weight is closely related to the health, growth and future productivity of a calf. For cattle, the last trimester of pregnancy is characterized by significant fetal growth, during which adequate nutrition is required to ensure adequate fetal growth. Therefore, studying placental angiogenesis is of great significance for increasing calf birth weights. Angiogenesis refers to the development of new blood vessels from existing capillaries or postcapillary veins, mainly including: degradation of vascular basement membrane in an activation phase; activation, proliferation and migration of vascular endothelial cells; and reconstruction to form new blood vessels and vascular networks, playing an important role during early embryonic development. Increasing angiogenesis can enhance placental blood flow and improve nutrient transfer from placenta to fetus, thereby increasing nutrient supply to the fetus, improving the metabolism of the fetus, and increasing the calf birth weight.

The small G protein Rap belongs to the Ras family, and RAP2C is a member of the small G protein Rap1, and a member of Rap including Rap1a, Rap1b, Rap2a and Rap2b. In addition, RAP2C is a small GTP binding protein that cycles between a GDP-bound inactive form and a GTP-bound active form, and can act as a molecular switch to regulate the proliferation, differentiation and apoptosis of cells. Studies in mice demonstrated that RAP2C, a gene upstream of the PI3K-Akt signaling pathway, could activate PI3K to further phosphorylate AKT, thereby affecting the proliferation of trophoblast cells and affecting angiogenesis. In addition to this, a study by Jiang et al. showed that in mice with overexpression of RAP2C, the activation of mammalian target of rapamycin complex 1 (mTORC1) might be promoted by increasing the phosphorylation level of protein kinase B (Akt), resulting in activation of sterol regulatory element-binding protein (SREBP) nuclear translocation and gene transcription, thereby regulating cholesterol biosynthesis, and the cholesterol biosynthesis is closely related to angiogenesis, as compared to that in normal mice. A study of human vascular endothelial cells by Doebele et al. found that either binding of Rap1 to a CAMP analog 8CPT by Epac or introduction of CA-Rap1 could inhibit Id1 (pro-angiogenic molecule) expression and induce TSP1 (anti-angiogenic molecule) expression, thereby promoting TSP1 to trigger the signaling cascade of cell surface receptors CD36 and CD47, mediating anti-angiogenic effects in endothelial cells, and inhibiting angiogenesis. In addition, a study in mice by Guba et al. found that RAP, an mTOR inhibitor having immunosuppressive activity, inhibited angiogenesis by reducing the production of vascular endothelial growth factor (VEGF) and significantly inhibiting the response of endothelial cells to VEGF stimulation. Furthermore, by injecting RAP into atherosclerotic rabbits, Chen et al. found that RAP could significantly inhibit angiogenesis in plaques, while in vitro assays of human umbilical vein endothelial cells (HUVECS) showed that RAP had an inhibitory effect on the angiogenic capacity of HUVECs. The above studies showed that RAP2C could play an important role in regulating angiogenesis by regulating certain biological processes or vascular factors involved in angiogenesis.

Blcattle black cattle is the first new germplasm in beef cattle successfully cultivated by somatic cell nuclear transplantation in China, where through techniques of cross breeding and molecular marker-assisted breeding, Luxi yellow cattle is improved using the semen from a breeding bull of black hair Wagyu to enable offspring to show a combination of excellent characteristics. The cattle is tall and muscular, and has prominent tendons, thin skin, thin bone, strong physique and well-proportioned structure. The beef of Blcattle black cattle is tender, tasty and rich in protein, has the amino acid composition closer to the needs of human body than that of beef, can improve the disease resistance of the body, is rich in iron content, and low in saturated fatty acid content and high in unsaturated fatty acid content in intermuscular fat, and has unique flavor. In 2015, the breed was recognized by experts as a new population, and was an excellent cattle breed for the production of marbled beef. Therefore, further optimizing the germplasm resources of Blcattle black cattle is of great significance for further popularizing the breed. In particular, calf birth weight is an indispensable part of target traits of cattle breeding, which not only reflects the growth and development status of calves at the embryonic stage, but also is closely related to the health, growth and future productivity of calves. By improving calf birth weight, the growth and development of calves can be promoted, the production performance of calves in the future can be improved, it is of great significance in calf breeding, and a feasible way to speed up the breeding of Blcattle black cattle is provided.

### SUMMARY

To solve the problems existing in the conventional technology, the present disclosure provides a method for screening high-quality calves of Blcattle black cattle by means of RAP2C, in which in the use of RAP2C in the regulation of the proliferation, differentiation and expression of placental vascular endothelial progenitor cells, RAP2C promotes angiogenesis and increases placental nutrient transport efficiency, thereby affecting calf birth weight.

To achieve the above objective, the present disclosure uses the technical solutions of: Use of RAP2C gene (Small G-Protein) in the breeding of Blcattle black cattle,
where the use includes screening individuals having a high RAP2C content as breeding cattle.

Use of RAP2C in the regulation of the proliferation and differentiation of bovine endothelial progenitor cells,
where the use includes overexpressing or knocking down or knocking out the RAP2C gene in bovine endothelial progenitor cells.

A method for preparing bovine endothelial progenitor cells, including the steps of:
(1) isolating and obtaining endothelial progenitor cells from bovine tissue;
(2) transfecting the endothelial progenitor cells with a RAP2C gene overexpression plasmid;
(3) screening and obtaining endothelial progenitor cells with regulated RAP2C gene expression.

The present disclosure has the following advantages:

In the present disclosure, it is proved by means of experiments that RAP2C can regulate placental angiogenesis in Blcattle black cattle and affect placental nutrient transport efficiency, thereby regulating calf birth weight. In breeding, individuals having a high RAP2C content should be screened as high-quality calves of Blcattle black cattle. The present disclosure provides a new direction for the breeding of high-quality beef cattle of Blcattle black cattle.

### BRIEF DESCRIPTION OF THE DRAWINGS

FIGS. 1A-1B show volcano plot (1A) and PCA plot (1B) of differential genes;

FIGS. 2A-2E show a weighted gene co-expression network analysis of differential genes and calf birth weight;

FIGS. 3A-3D show GO analysis (3A-3C) and KEGG enrichment analysis (3D) of differential genes;

FIG. 4 shows a diagram of RAP2C protein interaction network;

FIG. 5 shows analysis of association between RAP2C and calf birth weight;

FIGS. 6A-6C show analysis of mRNA (6A) and protein (6B-6C) expressions of RAP2C in different tissues;

FIGS. 7A-7C show analysis of mRNA (7A) and protein (7B-7C) expressions of RAP2C in Large calf placenta tissue and Small calf placenta tissue;

FIG. 8 shows identification of placental vascular endothelial progenitor cells;

FIGS. 9A-9F show the transfection efficiency of RAP2C plasmids;

FIGS. 10A-10B show the effect of RAP2C gene expression on invasion of placental vascular endothelial progenitor cells;

FIGS. 11A-11B show the effect of RAP2C gene expression on migration of placental vascular endothelial progenitor cells;

FIGS. 12A-12B show the expression level of CD31 in different treatment groups as detected by immunofluorescence assay;

FIGS. 13A-13I show the effect of different treatment groups on mRNA (13A-13D) and protein (13E-13I) of differentiation-related genes;

FIGS. 14A-14B show the proliferation of placental vascular endothelial progenitor cells in different treatment groups as detected by EDU;

FIGS. 15A-15I show the effect of different treatment groups on mRNA (15A-15D) and protein (15E-15IB) of proliferation-related genes;

FIGS. 16A-16B show the number of blood vessels in different treatment groups.

### DETAILED DESCRIPTION OF THE EMBODIMENTS

The present disclosure will be further described with reference to examples and drawings, but the present disclosure is not limited by the examples described below.

### Example 1 Screening of Calf Birth Weight-Related Differential Genes of Blcattle Black Cattle

### 1. Construction of Calf Phenotype and Transcriptome Library of Blcattle Black Cattle

Blcattle black cattle (cows) were selected from Shandong Zhaofu Animal Husbandry Technology Co., Ltd. The placental tissues were collected from 10 healthy Blcattle black cattle (cows) in similar feeding environment. Placentas were collected within 1 h after delivery of Blcattle black cattle (cows), and divided into two groups, namely Large calf placenta (LCP) group and Small calf placenta (SCP) group according to calf birth weights, with five placentas per group. After slaughter, the heart, liver, spleen, lung, kidney and skeletal muscle tissues were collected, placed in 5 mL cryopreservation tubes, and stored in liquid nitrogen for later use.

TABLE 1
Blcattle black cattle phenotype data in LCP group and SCP group

| | Large calf placenta | Small calf placenta |
|---|---|---|
| Indicator | group (LCP group) | group (H group) |
| Gestation period of cow, day | 281.00 ± 1.00^{a} | 280.00 ± 0.71^{a} |
| Calf birth weight, Kg | 33.80 ± 1.53^{A} | 30.30 ± 1.51^{B} |
| Body height, cm | 68.00 ± 0.84^{A} | 64.00 ± 0.45^{B} |
| Body length, cm | 59.20 ± 0.86^{A} | 54.90 ± 0.64^{B} |
| Chest circumference, cm | 78.00 ± 0.71^{A} | 72.20 ± 0.86^{B} |
| Waist circumference, cm | 81.00 ± 0.71^{A} | 75.80 ± 0.58^{B} |

Note:
Data are expressed as mean ± standard error, the same lowercase letter in the same line indicates no significant difference (P > 0.05), and different uppercase letters in the same line indicate extremely significant difference (P < 0.01).

The placental tissue samples were collected, the total RNA was extracted from the sample, and the RNA sample having good integrity and high purity was sent to GENEDENOVO for sequencing. With regard to the samples, the rRNA was removed from RNAs by using a conventional kit, the mRNA in RNAs was enriched, the enriched mRNA formed a double-stranded cDNA by using a reverse transcription system, the double-stranded cDNA was purified, end repair, addition of A to the end and ligation of a sequencing linker were performed, the cDNA of about 200 bp in length was screened using AMPURE XP beads for amplification, the PCR product was purified using AMPURE XP beads, and finally a transcriptome library was obtained.

### 2. Screening of Differential Gene

### 2.1 Analysis of Differential Expression

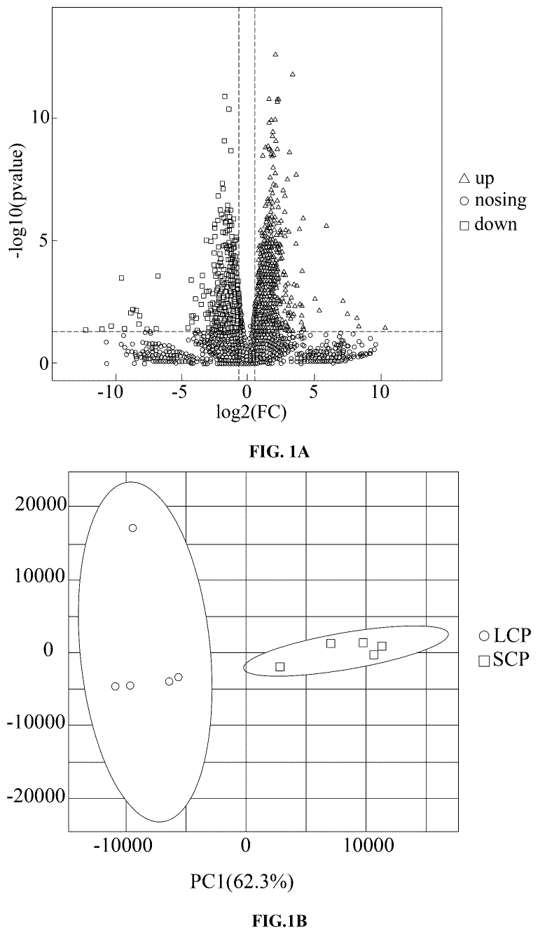

Differentially Expressed Genes (DEGs) for LCP and BCP were determined using DEseq2 software, with P-value≤0.05 and |Fold Change|≥1.5 as screening criteria. The analysis of the volcano plot (FIG. 1A) of DEGs finds that there are a total of 2743 DEGs between the two groups, with 1600 gene having up regulated expression and 1143 gene having down regulated expression in LCP. The analysis of the PCA plot (FIG. 1B) shows that the five biological samples from the same group are clustered together, indicating that the biological repeatability among the samples used in the study is good.

### 2.2 Weighted Gene Co-Expression Network Analysis (WGCNA)

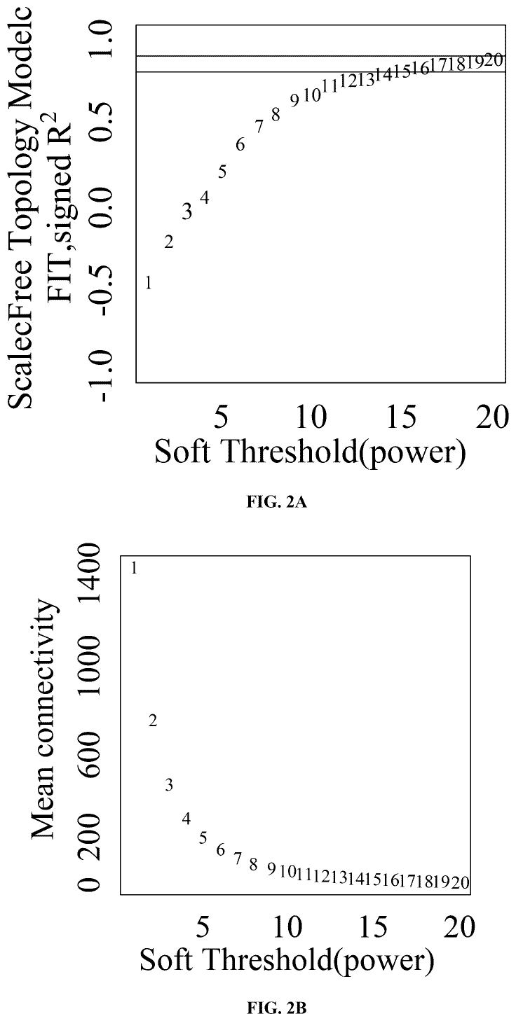

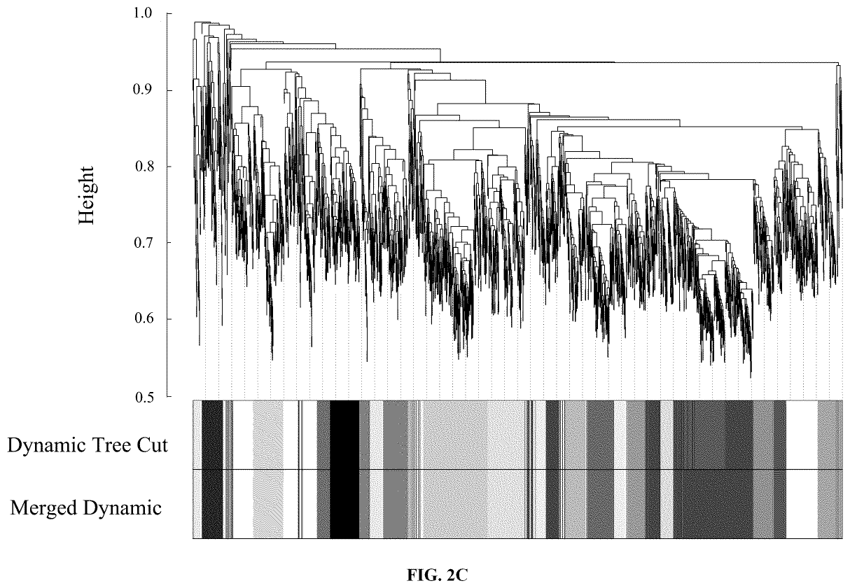

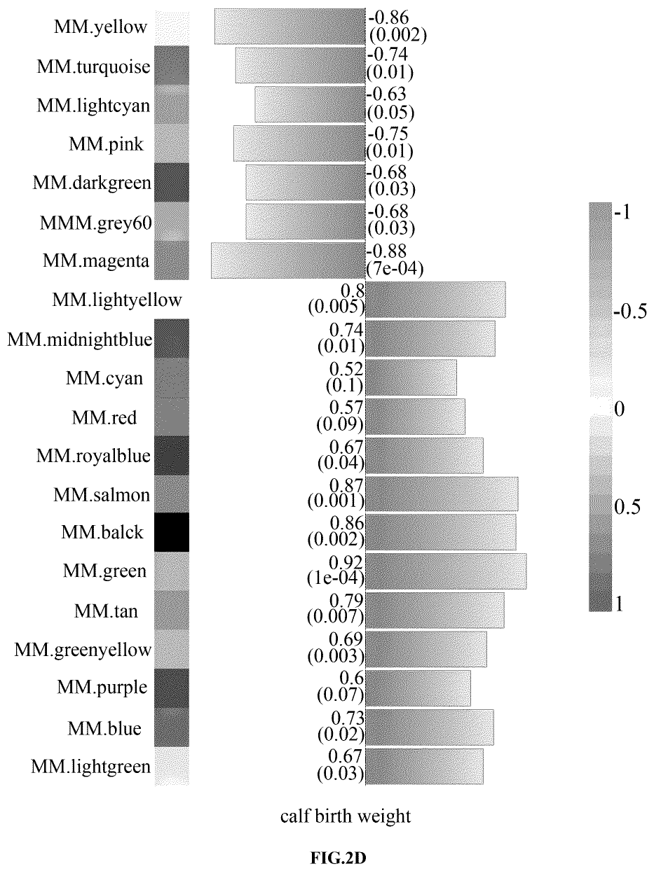

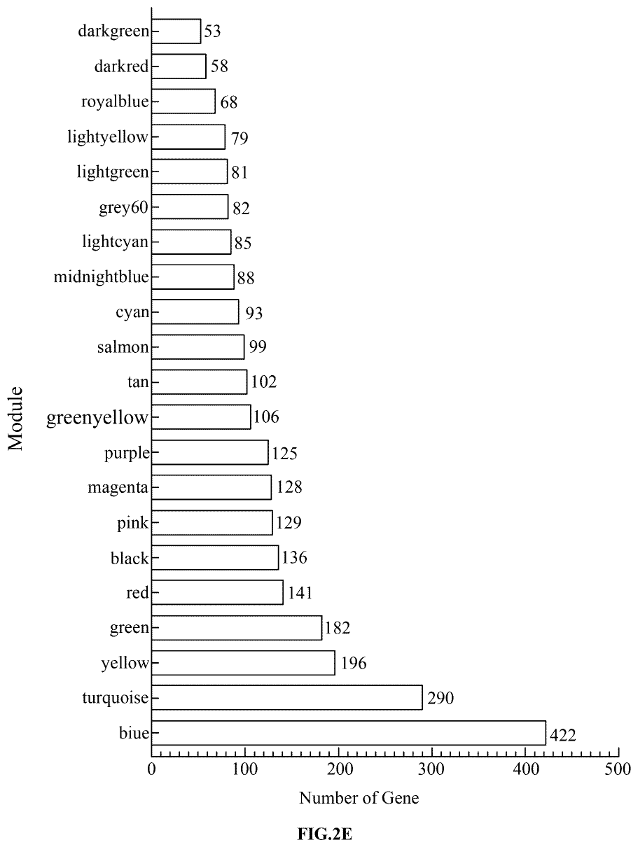

2743 DEGs were subjected to weighted gene co-expression with calf birth weight. The results in FIGS. 2A-2E show that a total of six gene co-expression modules associated with calf birth weight are identified, namely green (r=0.92, P=1e-04), magenta (r=−0.88, P=7e-04), lightyellow (r=0.8, P=0.005), salmon (r=0.87, P=0.001), black (r=0.86, P=0.002) and yellow (r=−0.86, P=0.002), where the module green has an association coefficient up to 0.92 with calf birth weight, and therefore the genes within the panel are selected as a key gene cluster.

### 2.3 GO and KEGG Enrichment Analyses

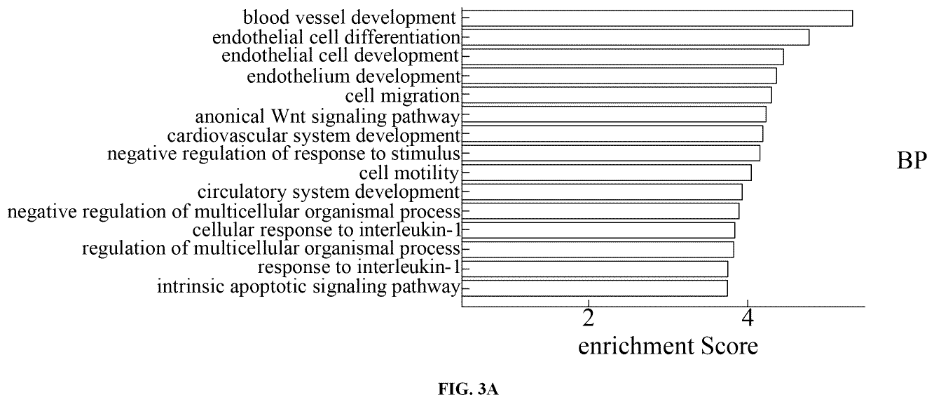

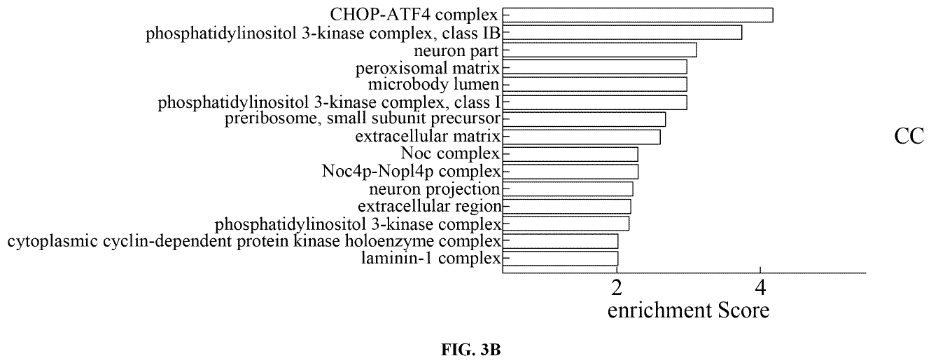

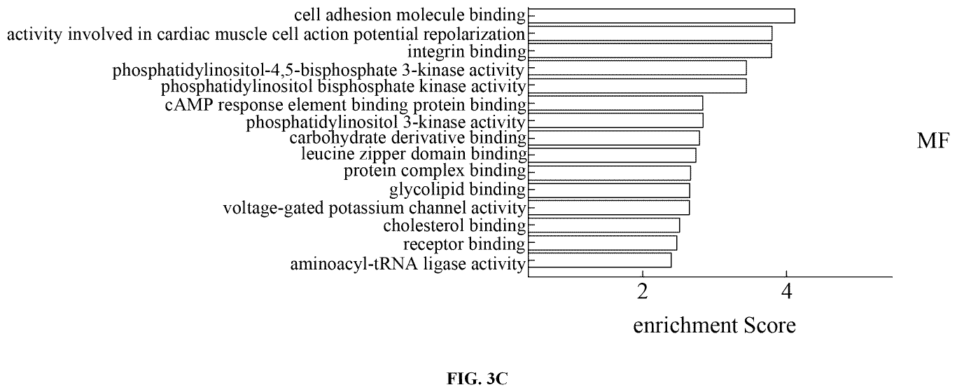

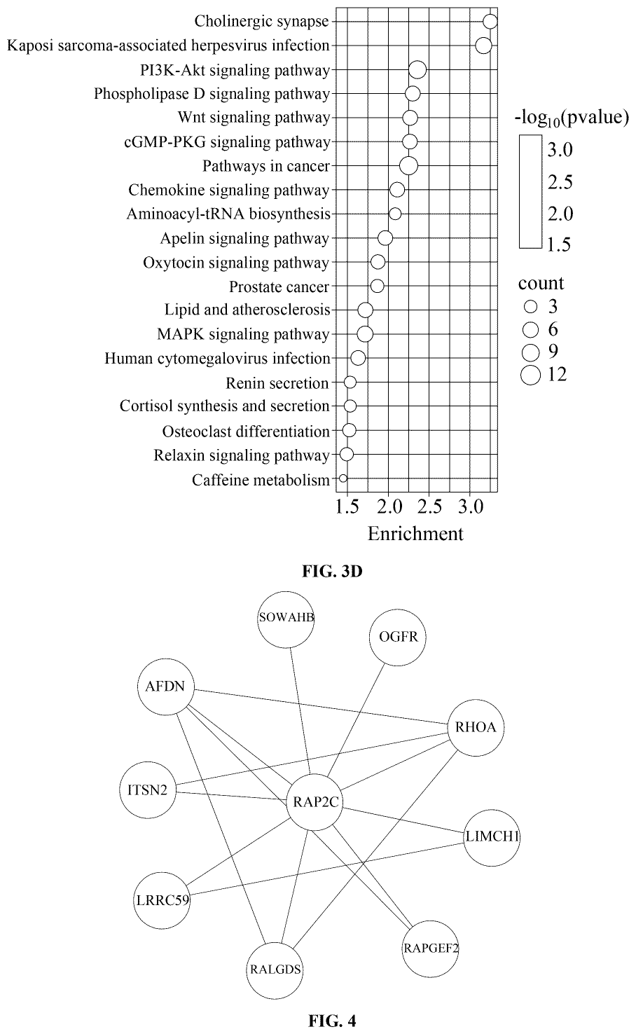

To identify the potential biological function of DEGs within the module green, GO and KEGG enrichment analyses were performed using R-package clusterProfiler. GO Terms can be divided into three categories, including: Biological Process (BP), Cellular Component (CC) and Molecular Function (MF). The threshold for significantly enriched GO Terms and KEGG pathways was set at FDR<0.05. The results in FIGS. 3A-3D show that DEGs are significantly enriched in biological processes such as vascular development, endothelial cell differentiation, and endothelial cell development, significantly enriched in cellular components such as phosphatidylinositol 3-kinase complex, extracellular matrix, and laminin-1 complex, and significantly enriched in molecular functions such as cell adhesion molecule binding, integrin binding, and phosphatidylinositol bisphosphate kinase activity. The KEGG enrichment results show that DEGs are mainly enriched in angiogenesis-related pathways (PI3K-Akt signaling pathway, Wnt signaling pathway, MAPK signaling pathway, etc.).

### 3. Functional Analysis of RAP2C and Identification of Expression Profiles in Tissues

### 3.1 Analysis of Protein Interaction Network

A protein interaction network for RAP2C was constructed using the STRING database (https://string-db.org/), and then visually displayed using Cytoscape 6.1 software. The results in FIG. 4 show that there are nine proteins that interact directly with RAP2C, namely LIMCH1, LRRC59, RAPGEF2, AFDN, RALGDS, RHOA, ITSN2, OGFR and SOWAHB, where the molecular functions of LRRC59, RAPGEF2 and RhoA are related to the proliferation and migration of vascular endothelial cells, affecting angiogenesis, and therefore RAP2C is screened as a candidate gene for angiogenesis.

### 3.2 Regression Analysis

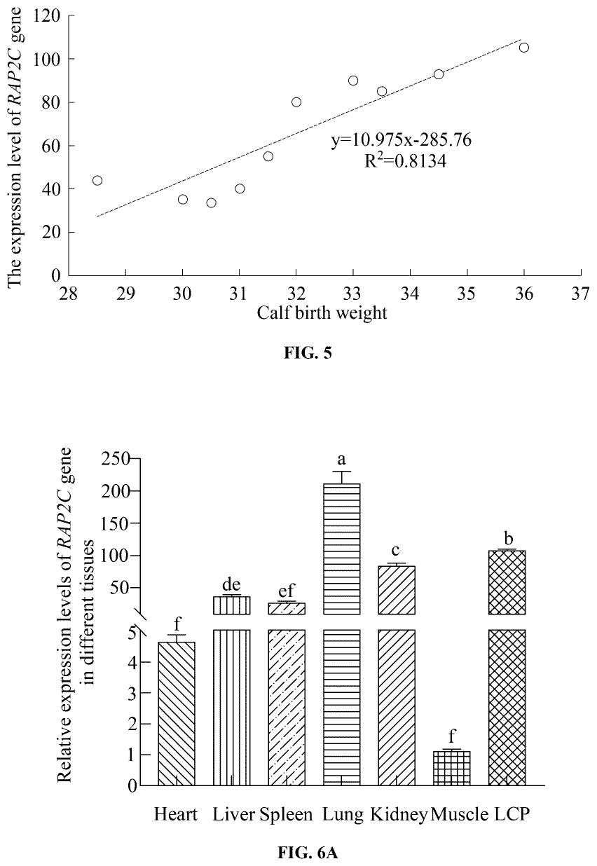

The expression level of RAP2C gene and the data of calf birth weight were fitted by using EXCEL software, and the fitting degree between them was analyzed. The results in FIG. 5 show that the RAP2C and calf birth weight regression agenda equation is y=10.975x−285.76, the fitting square is 0.81, the fitting degree is high, and with the increase of the gene expression level, the calf weight also increases, suggesting that RAP2C may positively regulate the calf birth weight.

### 3.3 Identification of Expression Profiles in Different Tissues, Large Placenta and Small Placenta Tissue

The placenta tissues from high calf birth weight group and low calf birth weight group (five placentas per group), as well as the heart, liver, spleen, lung and muscle were collected, and stored in liquid nitrogen. The total RNA was extracted using Trizol reagent (Invitrogen), and cDNA was obtained by reverse transcription. Taking the cDNA as a template, SYBR Green qPCR was performed on RAP2C gene. Glyceraldehyde-3-phosphate dehydrogenase (GAPDH) was used as the internal reference gene, the CT values of each sample were detected, the mean value was calculated, and the relative expression levels of RAP2C gene in each tissue were calculated. At the same time, each tissue protein was extracted using RIPA lysis liquid, the concentration of the extracted tissue protein was determined using a BCA kit, a western blot assay was performed using B-actin as the internal reference gene, and the relative expression levels of RAP2C protein in each tissue were calculated.

TABLE 2
qPCR primers for RAP2C and GAPDH

| | | SEQ ID |
|---|---|---|
| Gene | Primer sequence (5′→3′) | NO: |
| RAP2C | F: TCTTCCCTACCCGAGAAGCAA | 1 |
| | R: CTCCAAAGCAGCATCTGTGCAA | 2 |
| GAPDH | F: GATGCTGGTGCTGAGTATGT | 3 |
| | R: GCAGAAGGTGCAGAGATGAT | 4 |

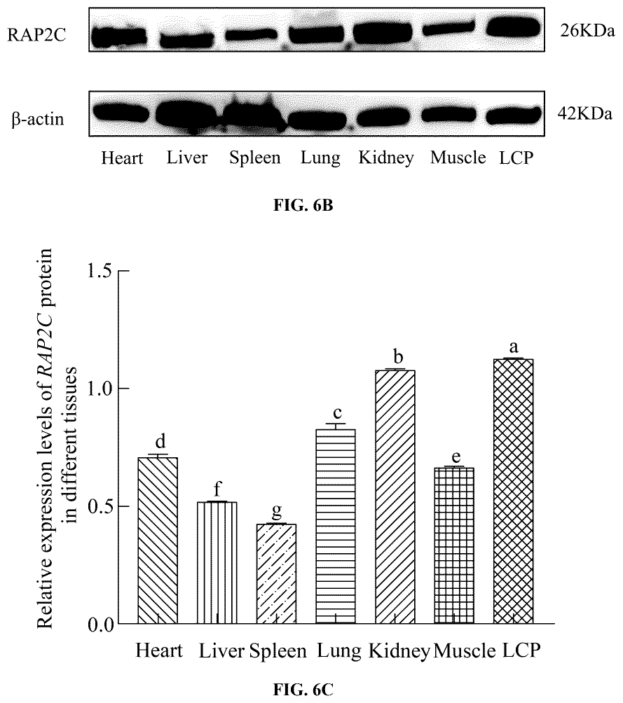

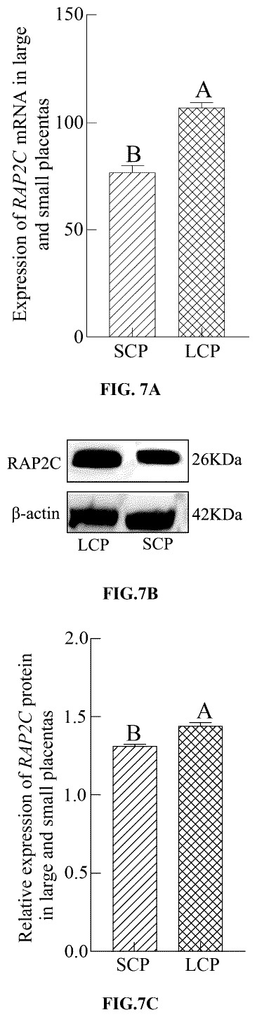

The analysis of mRNA (A) and protein (B) expressions of RAP2C in different tissues in FIGS. 6A-6C shows that the RAP2C gene is expressed to varying degrees in the heart, liver, spleen, lung, kidney, muscles and placenta of Blcattle black cattle, with a significantly higher expression level in the placenta than that in the other tissues (P<0.05), and that the expression level of RAP2C protein in the placenta is the highest, significantly higher than that in the other tissues (P<0.05), the expression level in the kidney is the second highest, and the expression level in the spleen is the lowest. The analysis of mRNA (A) and protein (B) expressions of RAP2C in Large/Small calf placenta tissue in FIGS. 7A-7C shows that the RAP2C gene is expressed in both Large calf placenta and Small calf placenta, with the expression level in Large calf placenta extremely significantly higher than that in Small calf placenta (P<0.01), and that the protein expression level is consistent with the gene expression level.

### Example 2 Construction of Placental Vascular Endothelial Progenitor Cells with Differential Expression of RAP2C

### 1. Culture and Identification of Placental Vascular Endothelial Progenitor Cells

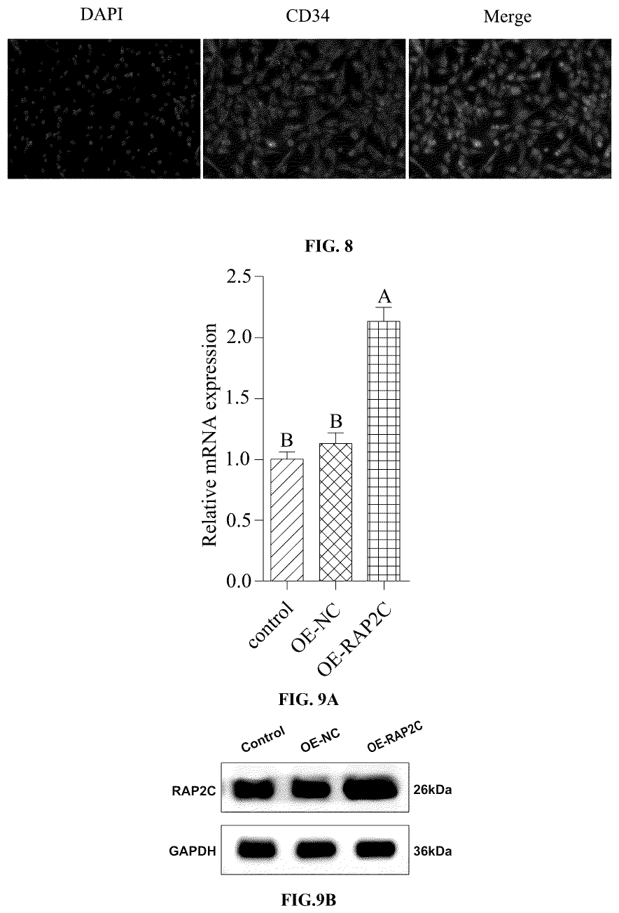

Bovine primary cells were isolated and cultured from the placenta tissue of newly born Blcattle calf, and made into a cell slide, and the cell slide was fixed with 4% paraformaldehyde for 15 min, permeabilized with 0.5% Triton X-100 for 2 h, and blocked with a blocking liquid for 2 h; added with a CD34 antibody and incubated overnight at 4° C.; added with a fluorescent secondary antibody and incubated for 2 h at 37° C.; added with DAPI and incubated for 5 min in the dark; and the slide was sealed with a Fluoromount-G mounting medium, and CD34 and DAPI fluorescence signals were collected. The results (FIG. 8) show that CD34, a marker for vascular endothelial progenitor cells, is expressed on the cells, indicating that the isolated cells are placental vascular endothelial progenitor cells.

2. Construction of Placental Vascular Endothelial Progenitor Cells with Differential Expression of RAP2C

On the basis of the mRNA sequence of RAP2C (GenBank No. NM_001075700.1), an overexpression plasmid (RAP2C-OE) and interference plasmids (siRNA RAP2C) were constructed. The sequences are as shown in Table 3.

TABLE 3
Sequences for constructing placental vascular
endothelial cells with differential expression
of RAP2C

| | | SEQ ID |
|---|---|---|
| Group | Sequence (5′→3′) | NO: |
| RAP2C-OE | F: GGACAGATTACACCAGCCCT | 5 |
| | R: CCTTCGCCCTTCACACAAAG | 6 |
| SIRNA | F: GCAAAGAGAUCGAAGUGGACUTT | 7 |
| RAP2C-120 | | |
| | R: AGUCCACUUCGAUCUCUUUGCTT | 8 |
| SIRNA | F: CACUGAUCCUAGUAGGAAAUATT | 9 |
| RAP2C-327 | | |
| | R: UAUUUCCUACUAGGAUCAGUGTT | 10 |
| SIRNA | F: GAGAAGCAAGAUCAAUGUUGUTT | 11 |
| RAP2C-509 | | |
| | R: ACAACAUUGAUCUUGCUUCUCTT | 12 |

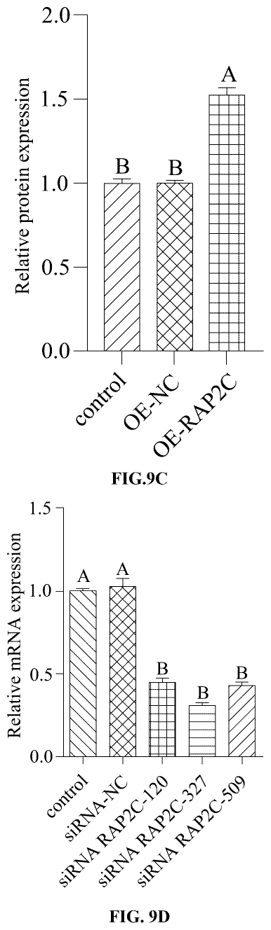

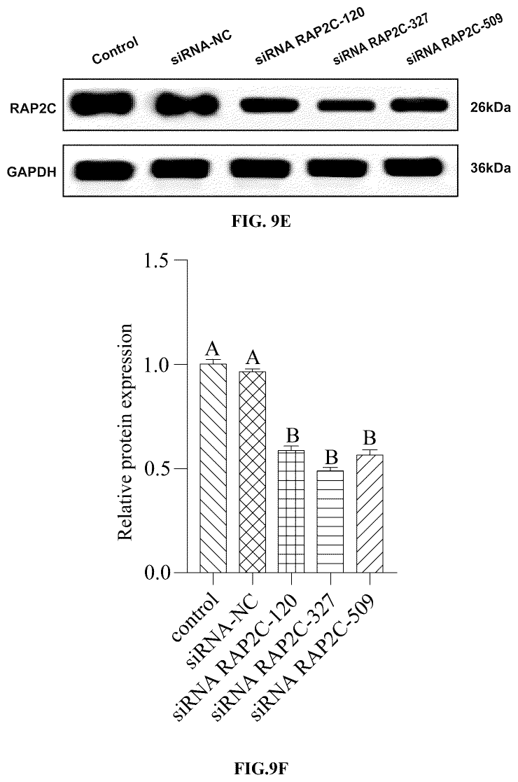

Normally cultured placental vascular endothelial progenitor cells were taken, and the original medium was aspirated. The cells were washed with PBS, and then digested with trypsin for 3 min, and then the digestion was terminated. The cell suspension was centrifuged at 200 g for 5 min. 20 μL of trypan blue was added to 20 μL of cell suspension for counting. The cells were plated at 3.5×10⁵ cells/well (6-well plate), with 2 mL per well. The cells were cultured at 5% CO₂, 37° C. overnight, and transfected with the overexpression plasmid and the interference plasmids, respectively. Screening was performed to obtain high RAP2C expression group cells and RAP2C interference group cells. After 24 h, the cells were collected for subsequent detection. The fluorescence quantification assay and western blot assay results (FIGS. 9A-9F) show that compared with the OE-NC group, the RAP2C-OE group has an extremely significantly increased expression level of RAP2C (P<0.01), and compared with the NC siRNA group, the siRNA RAP2C group has an extremely significantly decreased expression level of RAP2C (P<0.01). This indicates that the placental vascular endothelial progenitor cells of Blcattle black cattle are successfully transfected with the plasmids, and the transfected cells can be used for subsequent verification experiments; and the siRNA RAP2C-327 has the best interference efficiency and can be used for subsequent experiments.

### Example 3 Effect of RAP2C on Migration and Invasion of Placental Vascular Endothelial Progenitor Cells of Blcattle Black Cattle

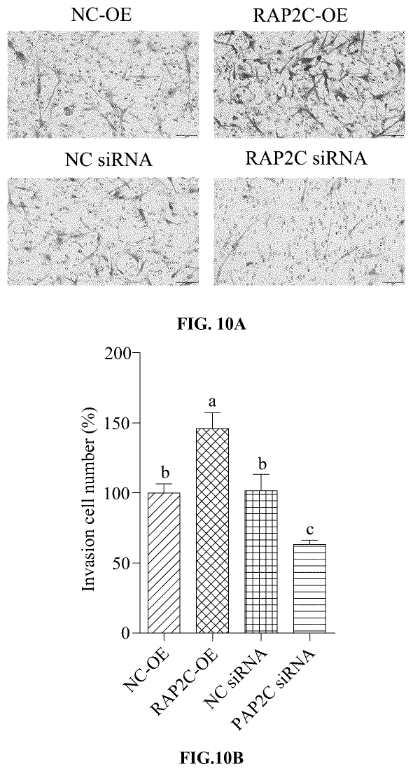

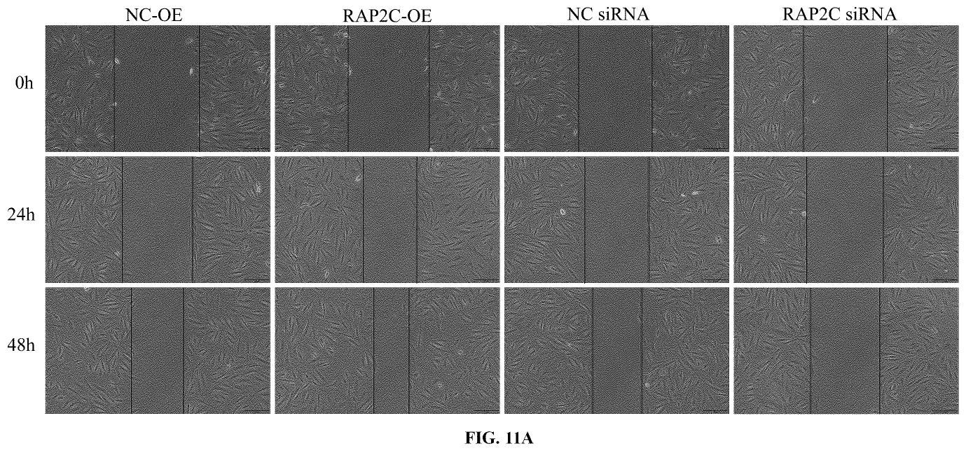

24 h after transfection of placental vascular endothelial progenitor cells with the overexpression/interference plasmids, the cells were collected for Transwell cell invasion detection: the cells were digested and centrifuged, the medium was discarded, and the cells were washed twice with PBS and adjusted to a cell density of 1.5×10⁵/mL; 200 μL of the cell suspension was added to a Transwell chamber, 600 μL of a medium containing 10% FBS was added to a lower chamber of a 24-well plate, and culture was performed at 37° C. for 24 h; the transwell chamber was taken out, the medium in the well was discarded, and the cells were washed twice with PBS, fixed with 4% PFA for 10 min, and washed twice with PBS; the cells were stained with crystal violet for 10 min, and washed twice with PBS, and the cells in the upper chamber were wiped away by using a cotton swab; and the invading cells were observed, photographed and counted by a microscope. The cell invasion results (FIGS. 10A-10B) show that high expression of RAP2C promotes the invasion of vascular endothelial progenitor cells, whereas the opposite result is present in the interference group.

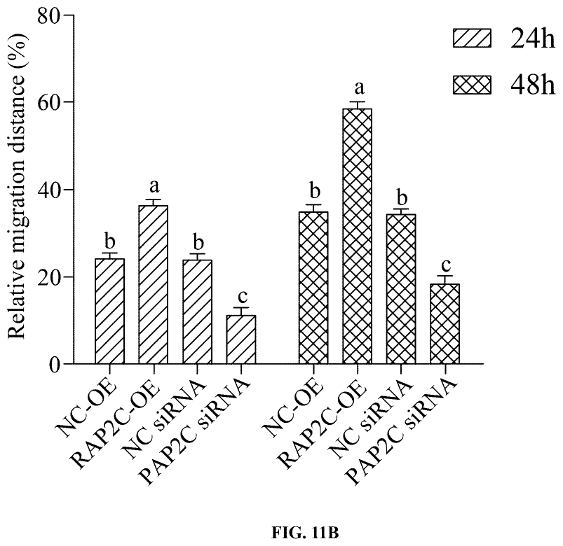

The placental vascular endothelial progenitor cells were transfected with overexpression/interference plasmids. After the placental vascular endothelial progenitor cells grew to 80%, a marker pen was used to draw a horizontal line mark on the back of a 6-well plate before the cells were seeded in the plate, a 10 μL pipettor tip was used to make cell scratches perpendicular to the plate, the cell medium was discarded, the cells was rinsed three times with PBS, added with a serum-free medium and the cells were taken out for photographing at 0 h, 24 h and 48 h of culture, respectively. The cell scratch results (FIGS. 11A-11B) show that high expression of RAP2C significantly promotes the migration of vascular endothelial progenitor cells, whereas the opposite result is present in the interference group.

### Example 4 Effect of RAP2C on Differentiation of Placental Vascular Endothelial Progenitor Cells of Blcattle Black Cattle

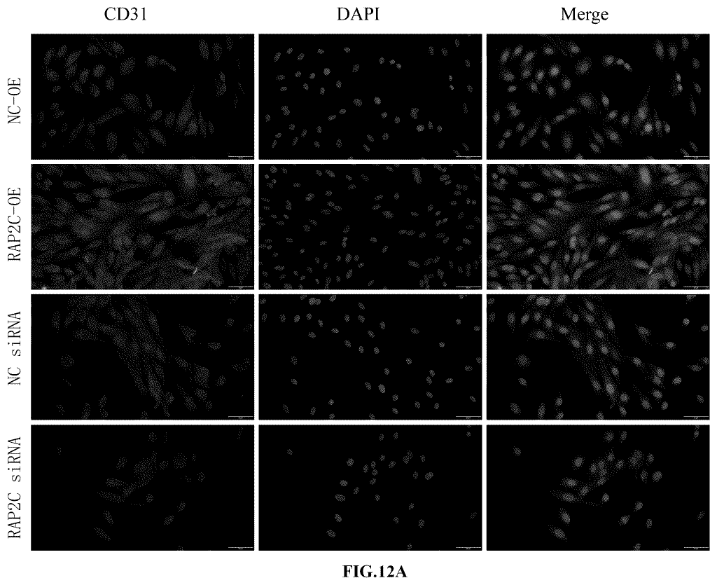

The placental vascular endothelial progenitor cells transfected with different plasmids were cultured in a medium containing vascular endothelial growth factor (VEGF, 50 ng/ml) and basic fibroblast growth factor (bFGF, 30 ng/mL) for 48 h, and then made into a cell slide, and the cell slide was fixed with 4% paraformaldehyde for 15 min, permeabilized with 0.5% Triton X-100 for 2 h, and blocked with a blocking liquid for 2 h; added with a CD31 antibody and incubated overnight at 4° C.; added with a fluorescent secondary antibody and incubated for 2 h at 37° C.; and added with DAPI, incubated for 5 min in the dark, the slide was sealed with a mounting medium containing an anti-fluorescence quencher, and CD31 and DAPI fluorescence signals were collected. The immunofluorescence results (FIGS. 12A-12B) show that the RAP2C-OE group has a significantly enhanced cell differentiation (P<0.05), and the RAP2C siRNA group has a significantly decreased cell differentiation (P<0.05), indicating that RAP2C promotes the differentiation of vascular endothelial progenitor cells.

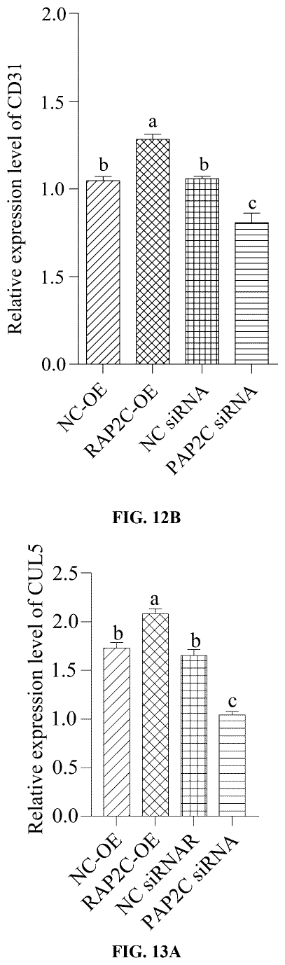

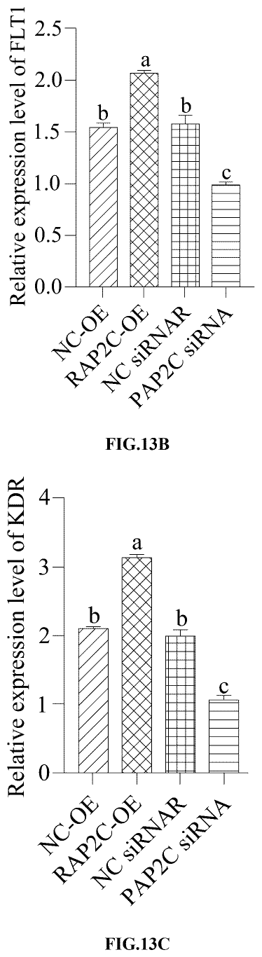

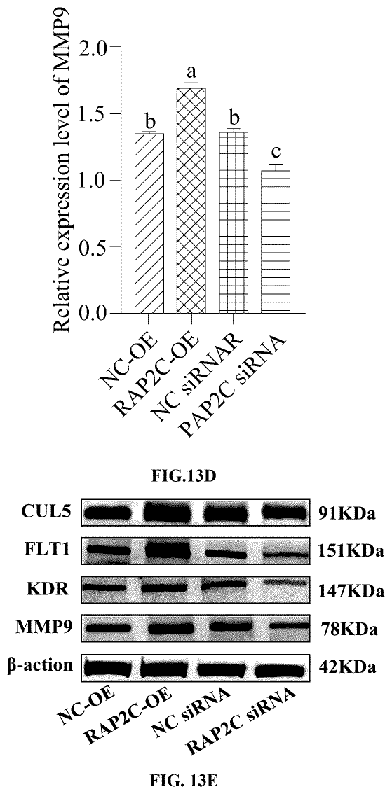

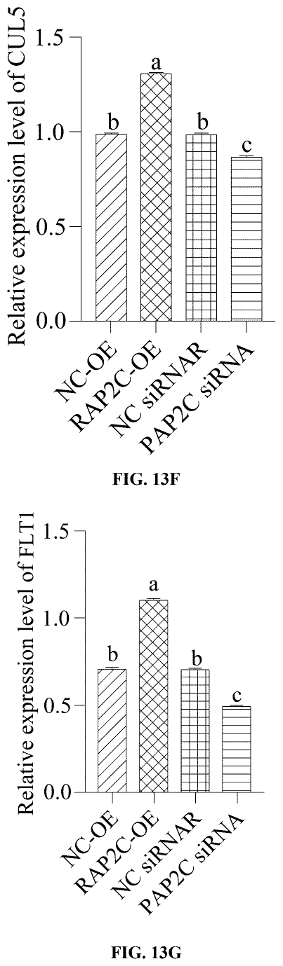

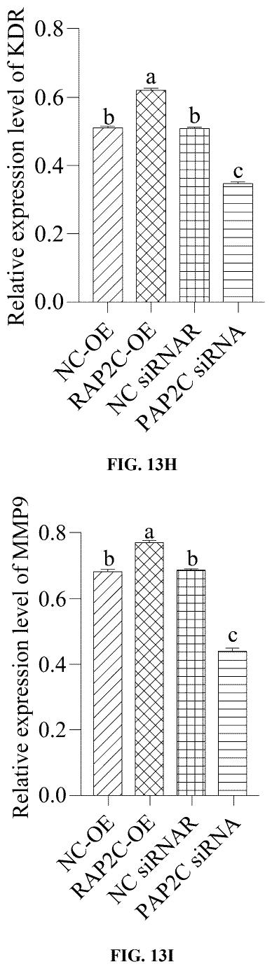

The placental vascular endothelial progenitor cells transfected with different plasmids were cultured in a medium containing vascular endothelial growth factor (VEGF, 50 ng/mL) and basic fibroblast growth factor (bFGF, 30 ng/mL) for 48 h, then the RNA and protein were extracted, and the changes in differentiation marker genes (CUL5, FLT1, KDR and MMP9) were detected. The results in FIGS. 13A-13I show that the RAP2C-OE group has significantly increased mRNA and protein levels of CUL5, FLT1, KDR and MMP9 (P<0.05), and the RAP2C siRNA group has significantly decreased mRNA and protein levels of CUL5, FLT1, KDR and MMP9 (P<0.05), indicating that RAP2C promotes the differentiation of vascular endothelial progenitor cells.

### Example 5 Effect of RAP2C on Proliferation of Placental Vascular Endothelial Progenitor Cells of Blcattle Black Cattle

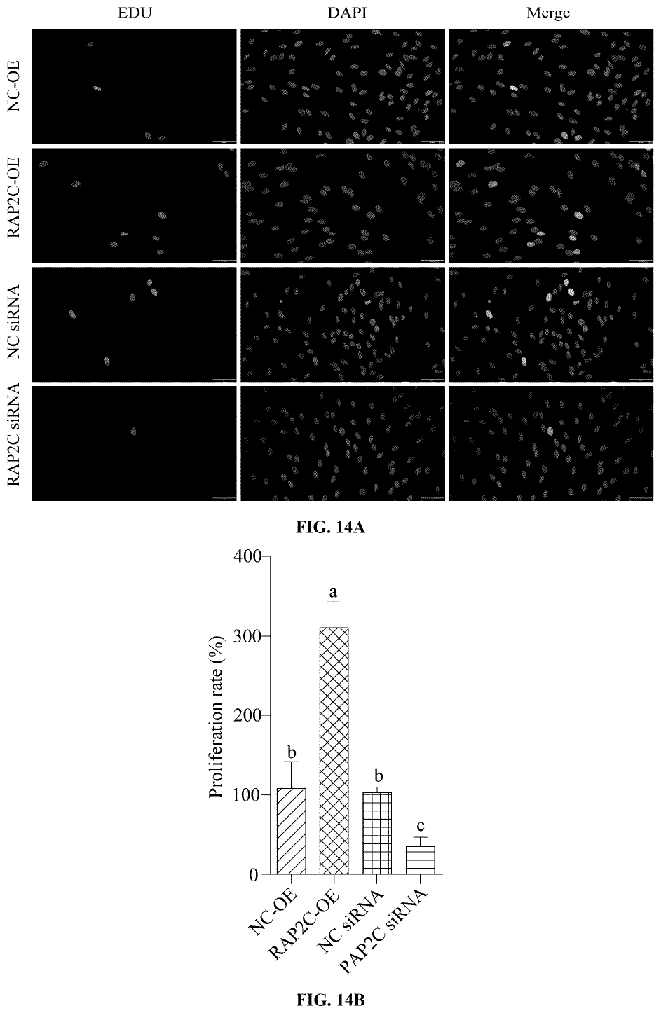

Normally cultured placental vascular endothelial progenitor cells were taken, and the medium was removed. The cells were rinsed with PBS, and digested with trypsin for 3 min, and then the digestion was terminated. The cell suspension was centrifuged at 200 g for 5 min, and resuspended with a medium. 20 μL of trypan blue was added to 20 μL of cell suspension for counting. The cells were plated at 45000 cells/well (24-well plate), and 500 μL per well was added. The cells were cultured at 5% CO₂, 37° C., and then transfected with the overexpression plasmid and the interference plasmids. The cells were incubated with EdU solution for 2 h. After washing with PBS, the cells were incubated with a cell fixative for 30 min, added with Glycine, washed with PBS, added with a permeant, washed with PBS, added with Apollo staining solution, incubated in the dark for 30 min, and the reaction solution was discarded. A permeant was added, washed and then discarded. A Hoechst reaction solution was added, incubation was performed in the dark for 30 min, and the solution was discarded. DAPI counterstain was performed, the slide was sealed and examined microscopically. The EdU positive signals were detected, and data analysis results were obtained. The EDU results (FIGS. 14A-14B) show that compared with the NC-OE group, the RAP2C-OE group has a significantly increased cell proliferation rate (P<0.05), indicating a significant increase in cells in S phase; and compared with the NC siRNA group, the RAP2C siRNA group has a significantly decreased cell proliferation rate (P<0.05), indicating a significant decrease in cells in S phase. It is fully demonstrated that RAP2C promotes the proliferation of the vascular endothelial progenitor cells of Blcattle black cattle.

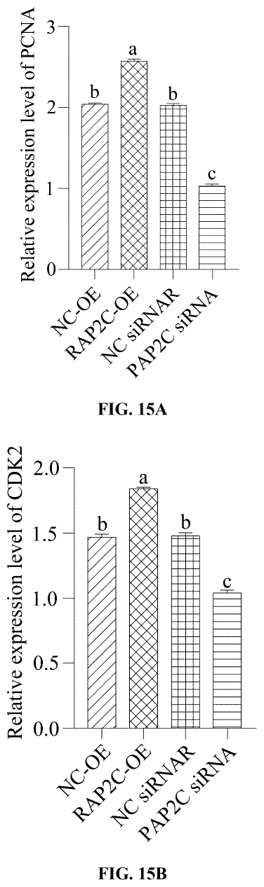

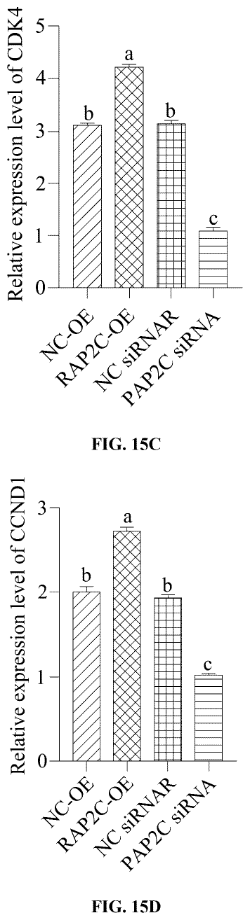

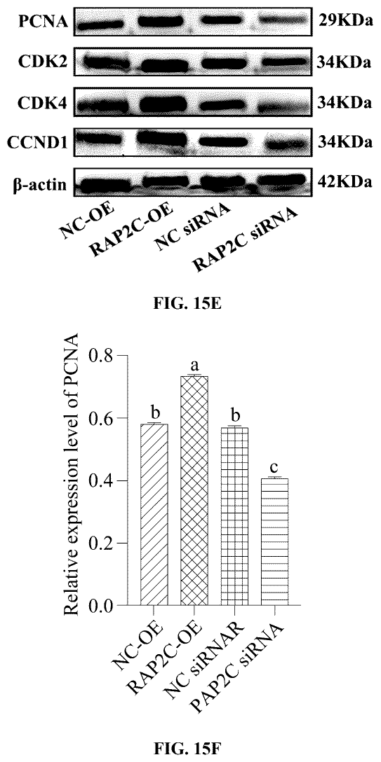

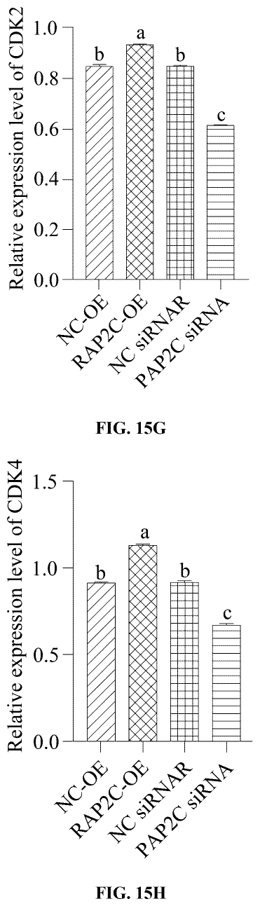

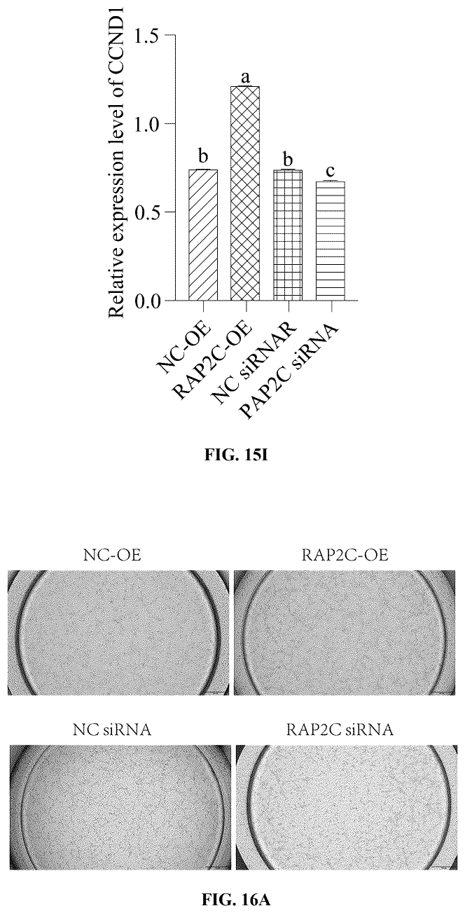

Normally cultured placental vascular endothelial progenitor cells were taken, and then transfected with the overexpression plasmid and the interference plasmids. After 48 h of culture in a medium, the RNA and protein were extracted, and the changes in proliferation marker genes (PCNA, CDK2, CDK4 and CCND1) were detected. The results in FIGS. 15A-15I show that the RAP2C-OE group has significantly increased mRNA and protein levels of PCNA, CDK2, CDK4 and CCND1 (P<0.05), and the RAP2C siRNA group has significantly decreased mRNA and protein levels of PCNA, CDK2, CDK4 and CCND1 (P<0.05), indicating that RAP2C promotes the proliferation of vascular endothelial progenitor cells.

### Example 6 Effect of RAP2C Gene Expression on Angiogenesis

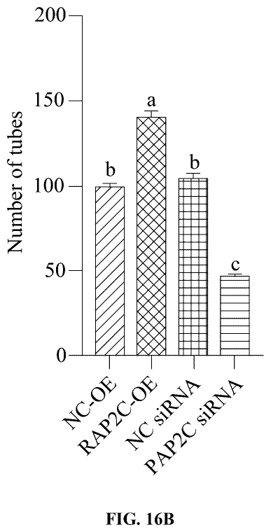

When the cell growth density reached 70%, the cells were plated in a 6-well plate at 3.5×10⁵ cells/well (6-well plate), with 2 mL per well. The cells were cultured at 5% CO₂, 37° C. overnight, and transfected with the RAP2C overexpression or interference plasmid. After 48 h, the medium was replaced with a medium containing vascular endothelial growth factor (VEGF, 50 ng/mL) and basic fibroblast growth factor (bFGF, 30 ng/mL) to induce the differentiation of vascular endothelial progenitor cells. 10 μL of Matrigel was added to each well of u-Slide, the u-Slide was placed in a 10 cm culture dish, added with 3 mL of sterile water, and placed for 30 min. Cell suspensions (NC-OE, RAP2C-OE, NC siRNA and RAP2C siRNA) were prepared, and were adjusted to a concentration of 2.5×10⁵ cells/mL after cell digestion. The u-Slide was taken out of the incubator, 50 μL of cell suspension was added to each well, and the culture dish was covered and then placed in the incubator. Culture was performed at 95% N₂+5% CO₂, 37° C. After 2 h, observation was performed, and the experimental results were recorded by taking photographs at appropriate time points. The Matrigel-based tube formation results (FIGS. 16A-16B) show that compared with the NC-OE group, the RAP2C-OE group has a significantly increase in the number of blood vessels (P<0.05); and compared with the NC siRNA group, the RAP2C siRNA group has a significantly decrease in the number of blood vessels (P<0.05), indicating that RAP2C promotes the formation of blood vessels.

The descriptions above are merely examples of the present application and are not used to limit the present application. For those skilled in the art, various changes and variations can be made to the present application. Any modifications, equivalent replacements, improvements, etc. made without departing from the spirit and principle of the present application shall fall within the scope of the claims of the present application.

What is claimed is:

1. A method for breeding Blcattle black cattle using RAP2C gene.

2. The method of claim 1, wherein individuals having a high RAP2C content are screened as breeding cattle.

3. A method for regulating the proliferation and differentiation of bovine endothelial progenitor cells using RAP2C gene.

4. The method of claim 3, wherein the RAP2C gene is overexpressed or knocked down or knocked out in bovine endothelial progenitor cells.

5. A method for preparing bovine endothelial progenitor cells, comprising the following steps:
(1) isolating and obtaining endothelial progenitor cells from bovine tissue;
(2) transfecting the endothelial progenitor cells with a RAP2C gene overexpression plasmid;
(3) screening and obtaining endothelial progenitor cells having up regulated RAP2C gene expression.


SEQUENCE LISTING (12 sequences)

SEQ ID NO: 1 - DNA, 21 bp, synthetic construct
  source 1..21: mol_type = other DNA; note = Primer RAP2C F
tcttccctacccgagaagcaa

SEQ ID NO: 2 - DNA, 22 bp, synthetic construct
  source 1..22: mol_type = other DNA; note = Primer RAP2C R
ctccaaagcagcatctgtgcaa

SEQ ID NO: 3 - DNA, 20 bp, synthetic construct
  source 1..20: mol_type = other DNA; note = Primer GAPDH F
gatgctggtgctgagtatgt

SEQ ID NO: 4 - DNA, 20 bp, synthetic construct
  source 1..20: mol_type = other DNA; note = Primer GAPDH R
gcagaaggtgcagagatgat

SEQ ID NO: 5 - DNA, 20 bp, synthetic construct
  source 1..20: mol_type = other DNA; note = Primer RAP2C-OE F
ggacagattacaccagccct

SEQ ID NO: 6 - DNA, 20 bp, synthetic construct
  source 1..20: mol_type = other DNA; note = Primer RAP2C-OE R
ccttcgcccttcacacaaag

SEQ ID NO: 7 - DNA, 23 bp, synthetic construct
  misc_feature 1..21: note = RNA
  source 1..23: mol_type = other DNA; note = siRNA RAP2C-120 F
  misc_feature 22..23
gcaaagagatcgaagtggacttt

SEQ ID NO: 8 - DNA, 23 bp, synthetic construct
  source 1..23: mol_type = other DNA; note = siRNA RAP2C-120 R
  misc_feature 1..21: note = RNA
  misc_feature 22..23: note = DNA
agtccacttcgatctctttgctt

SEQ ID NO: 9 - DNA, 23 bp, synthetic construct
  misc_feature 1..21: note = RNA
  source 1..23: mol_type = other DNA; note = siRNA RAP2C-327 F
  misc_feature 22..23: note = DNA
cactgatcctagtaggaaatatt

SEQ ID NO: 10 - DNA, 23 bp, synthetic construct
  misc_feature 1..21: note = RNA
  source 1..23: mol_type = other DNA; note = siRNA RAP2C-327 R
  misc_feature 22..23: note = DNA
tatttcctactaggatcagtgtt

SEQ ID NO: 11 - DNA, 23 bp, synthetic construct
  misc_feature 1..21: note = RNA
  source 1..23: mol_type = other DNA; note = siRNA RAP2C-509 F
  misc_feature 22..23: note = DNA
gagaagcaagatcaatgttgttt

SEQ ID NO: 12 - DNA, 23 bp, synthetic construct
  misc_feature 1..21: note = RNA
  source 1..23: mol_type = other DNA; note = siRNA RAP2C-509 R
  misc_feature 22..23: note = DNA
acaacattgatcttgcttctctt